(19) United States
(12) Patent Application Publication
(10) Pub. No.: US 2004/0199130 A1
(43) Pub. Date: Oct. 7, 2004

(54) Apparatus and method for treatment of macular degeneration

(76) Inventors: Victor I. Chornenky, Minnetonka, MN (US); Ali Jaafar, Eden Prairie, MN (US)

Correspondence Address: Law Offices, P.O. Box 386353, Bloomington, MN, 55438, US

(21) Appl. No.: 10/817,230

(22) Filed: Apr. 2, 2004

Related U.S. Application Data
(60) Provisional application No. 60/459,689, filed on Apr. 3, 2003.

(51) Int. Cl. A61M 35/00; A61K 31/205; A01N 37/30

(52) U.S. Cl. 604/289

(57) ABSTRACT

The present invention provides apparatus and method for treating macular degeneration. An apparatus has a therapeutic agent delivery system that is placed into proximity of the sclera of an eye affected by macular degeneration. A therapeutic agent or agents is then delivered to the sclera either by injection or diffusion, with the therapeutic agent being provided for the dissolution of waste products in Bruch's membrane. In a method a therapeutic agent delivery system is disposed in proximity of the sclera and one or more therapeutic agents are injected or diffused into the sclera to provide for the dissolution of waste products in Bruch's membrane.

The present application claims priority from U.S. Provisional Patent Application Serial No. 60/459,689, filed Apr. 3, 2003, and entitled Apparatus and Method for Treatment of Age-Related Macular Degeneration, the specification and drawings of which are incorporated herein in their entirety by reference.

### FIELD OF THE INVENTION

The present invention relates generally to apparatus and methods for treatment of ophthalmologic problems and specifically to apparatus and methods for the treatment of macular degeneration.

### BACKGROUND OF THE INVENTION

Macular degeneration is a chronic eye disease that occurs when tissue in the macula, the part of the eye that is responsible for central vision, deteriorates. This condition tends to develop as a person gets older with age being the highest risk factor for the development of macular degeneration. In fact, macular degeneration is the leading cause of severe vision loss in people age 50 and older. Hence, although the disease can strike younger people and even children on occasion, it is often referred to as age-related macular degeneration, or ARMD.

For a proper understanding of the disease, a basic knowledge of the structure of the eye is helpful. Thus, referring to FIGS. 5-6 and 9-11, an eye 10 is shown in an enlarged cross-section, with FIG. 10 illustrating a horizontal cross section of a patient's left eye.

The eye 10 includes a tough, outer layer of tissue known as the sclera 12 forming the vitreous cavity 14, which is filled with a fluid known as the vitreous humor. Sclera 12 is a sponge-like, strong protective layer of an eye comprising mainly laminated collagen fibers. Light enters the eye through the cornea 16, passes through the lens 18 and impinges on the retina 20. The retina 20 is made up several cell layers, including the light sensitive cells called rods and cones that receive the light and pass the signals onto the optic nerve 22, which carries the signals to the brain.

Several layers of tissue lie between the sclera 12 and the retina 20. Included among them are the retinal pigment epithelium (RPE) 24, which is a mono layer of cells interfacing the outer segments of the retina. Lying adjacent to the RPE 24 is a thin, non-cellular membrane 26 known as Bruch's membrane. Bruch's membrane 26 comprises several layers of collagen and elastin fibers connected into a mesh that is easily permeable for fluids and solvents diffusing to and from the RPE. Underlying Bruch's membrane 26 is the choroid 28, a well developed layer of fenestrated (perforated) capillaries providing nutrients for the RPE and the rods and cones of the retina and disposing of the metabolic wastes from them. The RPE 24 also serves as a blood-ocular barrier, preventing blood from leaking to the retina 20 and inside the vitreous cavity 14. That is, the apical domain of the retinal pigment epithelium 24 has tight junctions 34 (FIG. 6) that form the blood-ocular barrier, which prevents any uncontrollable diffusion of even small molecules through the apical domain of the RPE.

Also seen in the figures is an artery 30 and a vein 32 responsible for nourishing the eye and removing the waste products therefrom.

The central part of the retina 20 that is responsible for the best vision is the fovea 36. The area of the retina immediately surrounding the fovea 36 is the macula, generally indicated in the view by arrow 38. The fovea 36 and macula 38 form the region of the retina where the rods and cones are most densely packed. These cells, particularly the cones, are essential for central vision essential to such tasks as reading and driving a vehicle and are the ones affected by the degeneration of the macula 38.

There are several early symptoms of macular degeneration. Blurred vision is one. Another early symptom of macular degeneration may be a need for more light to do close-up work. Still another early symptom is that fine print may become harder to read and street signs may become more difficult to recognize. As the damage to the macula 38 increases, eventually a patient may notice that, when looking at an object, what should be a smooth, straight line appears instead to be distorted or crooked. Gray or blank spots may begin to mask the center of the visual field. Progression of the damage and the consequent reduction in vision may, and usually does, lead to severe vision loss in one or both eyes. The condition usually develops painlessly and gradually, though in some instances it may develop rapidly.

Macular degeneration affects central, but not peripheral vision; thus it doesn't cause total blindness. Still, the loss of clear central vision—critical for reading, driving, recognizing people's faces and doing detail work—greatly affects the quality of life. In tragically few cases is it possible to reverse even partially the damage caused by macular degeneration.

Macular degeneration occurs in two types: dry and wet macular degeneration. In either form of macular degeneration, a person's vision may falter in one eye while the other remains fine for years. Any or much change may not be noticed because the other good eye compensates for the weak one. The person's vision and lifestyle begin to be dramatically affected when this condition develops in both eyes. Depending on which of the two types of macular degeneration is developing, the signs and symptoms of the disease may vary.

Most people with macular degeneration have the dry form. In fact, macular degeneration always starts out as the dry form. The dry form may initially affect only one eye but, in most cases, both eyes eventually become involved. Dry macular degeneration occurs when the RPE cells begin to thin. The normally uniform reddish color of the macula 38 takes on a mottled appearance. Drusen, which look like yellow dots and are deposits of extracellular materials, appear under the retina. With dry macular degeneration the following symptoms may be noticed: the need for increasingly bright illumination when reading or doing close work, printed words that appear increasingly blurry, colors that seem washed out and dull, gradual increases in the haziness of the overall vision; and/or a blind spot in the center of the visual field combined with a profound drop in the central vision. Initially, in spite of these developments, little or no change may be noticed in vision. Many people who've received a diagnosis of early-stage dry macular degeneration may not be bothered with symptoms such as blurred eyesight unless they live to a very old age. But as the drusen and mottled pigmentation continue to develop, vision may deteriorate sooner. Thinning of the RPE may progress to a point where this protective layer of the retina disappears. This affects the overlying cones and rods and may result in complete loss of central vision.

While the dry form of macular degeneration accounts for 85-90 percent of all cases of macular degeneration, the wet form is responsible for nearly 90 percent of the severe vision loss that people with macular degeneration experience. If wet macular degeneration in one eye develops, the odds of getting it in the other eye increase greatly. With wet macular degeneration, the following symptoms may appear rapidly: visual distortions, such as straight lines appearing wavy or crooked; decreased central vision; and/or a central blurry spot. Sight loss is usually rapid and severe, and usually results in legal blindness, defined as 20/200 vision or worse. This means that what someone with normal vision can see from 200 feet, a person with 20/200 vision can see only from 20 feet.

All eyes with the wet form also show signs of the dry form—that is, drusen and mottled pigmentation of the retina. But eyes suffering from wet macular degeneration differ in that they grow new blood vessels from the choroid underneath into the macula 38. These vessels penetrate Bruch's membrane and leak fluid or blood—hence the name wet macular degeneration—into the retina and cause central vision to blur. This abnormal blood vessel growth is known as choroidal neovascularization, or CNV.

Wet macular degeneration, much like the dry form of macular degeneration, is believed to be caused by a breakdown in the nutrient/waste removal system. That is, when the waste from the cones and rods isn't disposed of and begins to accumulate, sufficient flow of nutrients to the macula 38 is disrupted. The abnormal growth of blood vessels characteristic of the wet form is believed to be a response to this disruption in the flow of nutrients. That is, without enough nutrients, healthy tissue in the macula 38 begins to deteriorate so the eye attempts to compensate for the disruption in nutrient flow caused by waste accumulation by growing additional blood vessels to enhance the nutrient flow to the macula 38. Stated otherwise, there is evidence that the growth of new blood vessels takes place as a response of the choroid to a biological signal of lack of oxygen released by the RPE. The carrier of the signal, the vascular endothelial growth factor (VEGF) actually is responsible for triggering growth of new blood vessel.

Treatment options for macular degeneration depend upon the form affecting the eyes. Currently there's no treatment for dry macular degeneration. Dry macular degeneration usually progresses slowly, so many people with this condition are able to live relatively normal, productive lives, especially if only one eye is affected.

Some treatment options are available for wet macular degeneration, however. All existing methods of treatment of wet macular degeneration are directed to the destruction of the choroidal neovascularization that destroys the patient's central vision. But the success of the treatment—stopping further progress of the disease—depends on the location and the extent of the abnormal blood vessels growth, or CNV, at the time of the treatment. In most cases the damage already caused by macular degeneration can't be reversed. The sooner CNV is detected, the better chances are of treatment preserving what's left of the central vision.

Treatments for wet macular degeneration, all of which can be done as outpatient procedures, include photocoagulation, photodynamic therapy, and macular translocation therapy.

Photocoagulation therapy. In photocoagulation therapy a doctor uses a high-energy laser beam to create small burns in areas with abnormal blood vessels. The process can seal off and destroy the CNV that has developed under the macula 38. The procedure can prevent further damage to the macula 38 and halt continued vision loss. Only about 20 percent of people who have wet macular degeneration are candidates for this procedure, however. The availability of photocoagulation as wet macular degeneration treatment depends on the location and appearance of the CNV, the amount of blood that has leaked, and the general health of the macula 38. Even if photocoagulation is a viable option for a particular patient, the results can be disappointing. Laser surgery to destroy the CNV is successful only about 50 percent of the time. And even successfully destroyed CNV has a tendency to recur. Repeat laser treatment may not be possible in such an event.

If a patient noticed a dark or gray spot in or near the central vision before laser treatment, the procedure will make vision in that spot completely and permanently blank. With time the patient may not notice the blank spot any longer, especially when the patient can use both eyes. Photocoagulation therapy is the only proven treatment for CNV when it's not located directly under the fovea 36 at the center of macula 38.

Photodynamic therapy (PDT). This therapy is useful for treating CNV that's located directly under the fovea 36. As noted earlier, the fovea 36 lies at the center of the macula 38 and in healthy eyes provides the sharpest vision. If conventional high-energy photocoagulation laser surgery were used at this location, it would destroy all central vision. PDT increases chances of preserving some of that vision.

PDT is a procedure that combines a low-energy laser and a light-sensitized drug that's injected into the bloodstream. The drug concentrates in the CNV under the macula 38. When the doctor directs the low-energy laser light at the macula 38, the drug absorbs the light and in response releases atomic oxygen that chemically attacks the abnormal blood vessels without damaging the macula 38, thus transforming the CNV into a thin scar. The overlying rods and cones are largely preserved, resulting in better vision than if the patient had had high-energy laser surgery or no treatment at all. The therapy can be repeated if the CNV doesn't close or if it reopens after initial closure.

The Food and Drug Administration has approved the drug verteporfin (Visudyne) for use in photodynamic therapy. Studies involving verteporfin demonstrate that over a 2-year period, multiple treatment sessions reduced vision loss for two-thirds of the people who had clearly defined CNV under the fovea 36. Though these results are promising, other long-term benefits are still under study. For example, further research will determine if this treatment also helps people who have poorly defined or hidden areas of CNV.

Macular translocation surgery. Macular translocation surgery is an experimental treatment for wet macular degeneration. This surgery can be used if the abnormal blood vessels are located directly under the fovea 36. In this procedure, a surgeon detaches the retina, shifts the fovea 36 away from the CNV, and relocates it over healthy tissue. When the CNV is exposed, the surgeon can then use a high-energy laser to destroy blood vessels without damaging the fovea 36. This surgery can be performed only if vision loss is recent (usually within 1 to 3 months), the extent of CNV is limited and the tissue around the fovea 36 is healthy.

Thus, while progress has been made in treating macular degeneration once it has developed, little has been done to prevent the development of the condition in the first instance. Development of a preventative therapy would be aided by an understanding of what are believed to be the root causes of the condition. Recently, studies presented by several research groups indicate that deposits of waste products in Bruch's membrane, and especially lipid deposits, may play a major role in breakdown of the nutrient/waste disposal mechanism and the consequent development of macular degeneration.

More specifically it is believed that with age the RPE may deteriorate and become thin (a process known as atrophy). This RPE atrophy impacts the ability of the RPE to perform its biological functions properly, a major one of which is to supply the retina 20 with nutrients coming from the choroid 28 and to remove waste products from the retina 20 to the choroid. This critical nutrient/waste two-way traffic occurs through Bruch's membrane 26. Thus, it is believed that RPE atrophy results in a declining efficiency of the nutritional and waste removing cycles between the retina 20 and the choroid 28. Consequently, waste deposits begin to form in Bruch's membrane 26 and the light-sensitive cells of the macula 38 become damaged due to a decline in nutrition. The deposits of wastes—lipids—progresses exponentially with age and substantially changes the diffusion characteristics of Bruch's membrane. Especially detrimental for the diffusion or transport of nutrients through the membrane are depositions of neutral lipids, or fats, that increase the membrane's hydrophobicity and consequently, resistance of the membrane to the transfer of fluids across it.

As the cells in the retina become progressively damaged, it is believed that their ability to send normal vision signals through the optic nerve 22 to the brain is progressively reduced. Generally speaking, it is believed that the fovea 36 in particular is the area where degeneration of the retina takes place and under which Bruch's membrane becomes clogged with metabolic wastes.

There is a need in developing an apparatus and a method of treatment of macular degeneration that would improve the diffusive characteristics of Bruch's membrane so as to improve the exchange of nutrients and waste disposal between the RPE and choroid and that preferably is minimally invasive.

### BRIEF DESCRIPTION OF THE INVENTION

An object of present invention is to provide treatment of Bruch's membrane to improve its diffusion properties.

Another object of present invention is to deliver medication into Bruch's membrane that will dissolve lipid deposits in the body of the membrane and assist in their removal through the choroidal circulation.

Still another object of the present invention is to provide apparatus and method for the treatment of macular degeneration.

These and other objects of the present invention are achieved by apparatus and method for delivering a natural enzyme lipase (lipoprotein lipase) into the posterior sclera in close proximity to the macula 38.

The present invention provides apparatus and method for treating macular degeneration. In accord with the invention, an apparatus may have a handle mounting an elongate, hollow probe having a proximal end attached to the handle and a distal end with an opening. The probe distal end preferably has a curved configuration to conform substantially with the shape of the eye. The probe houses a therapeutic agent delivery apparatus within the hollow interior or passage defined by the probe wall. The delivery apparatus is movable between a retracted or passive position wherein the delivery apparatus is disposed within the probe and an extended or active position wherein the delivery apparatus extends out from the distal probe end opening. The delivery apparatus is fluidly connected to a therapeutic agent reservoir. In use, after proper positioning of the probe distal end relative to the macula 38 and the area of the eye to receive therapy, the delivery apparatus will be extended so as to engage the sclera and lipase and/or other waste dissolving therapeutic agents will be provided to the sclera from the reservoir.

More generally, the present invention provides a therapeutic agent delivery system for providing lipid dissolving agents to the eye. An apparatus in accord with the invention will include a probe having a therapeutic agent dispensing opening through which the therapeutic agent is delivered to the eye generally, and the sclera in particular.

In one embodiment of the present invention, the delivery apparatus is an elongate needle movable between a retracted or passive position wherein the distal end of the needle is disposed within the probe and an extended or active position wherein the distal needle end extends out from the distal probe end opening. The needle is fluidly connected to a pharmaceutical reservoir. In use, after proper positioning of the probe distal end relative to the macula 38 and the area of the eye to receive therapy, the distal needle end will be extended so as to penetrate the sclera and lipase and/or other waste dissolving therapeutic agents, principally lipases, will be injected into the sclera from the reservoir. The lipase will or similar agent will dissolve the waste products accumulated in Bruch's membrane, which will allow them to be carried away by the bloodstream, thus clearing the membrane of such waste materials, restoring greater efficiency to the nutrient/waste cycle operating between the macula 38 and the choroid, and delaying or preventing the progression of the degeneration of the macula 38.

In another embodiment of the invention, the delivery apparatus be a micro-needle array fluidly connected to the reservoir, with the micro needles being extended into engagement with the sclera by the appropriate mechanism.

In another embodiment of the invention, the probe may provide a delivery apparatus taking the form of a porous pad fluidly connected to the reservoir that is disposed against the sclera during a therapy procedure and that enables the therapeutic agents to diffuse into the sclera.

In another embodiment of the invention, the probe may provide a delivery apparatus taking the form of a plurality of porous pads, with at least a pair of the pads being electrically connected to an electric power source to enhance the diffusion of the therapeutic agents into the sclera by means of iontophoresis.

In a method in accord with the present invention a therapeutic agent delivery system is provided and disposed adjacent to the sclera of an eye affected by macular degeneration. One or more therapeutic agents, principally lipases, are injected or diffused into the sclera to provide for the dissolution of waste products in Bruch's membrane.

The foregoing objects and features of the present invention, as well as other various features and advantages, will become evident to those skilled in the art when the following description of the invention is read in conjunction with the accompanying drawings as briefly described below and the appended claims. Throughout the drawings, like numerals refer to similar or identical parts.

### BRIEF DESCRIPTION OF THE DRAWINGS

FIG. 1 illustrates an embodiment of a therapeutic agent delivery system for macular degeneration therapy in accord with the present invention.

FIG. 2 represents the distal end of the embodiment shown in FIG. 1 taken along viewing plane A-A of FIG. 1.

FIG. 3 shows another version of the distal end of the embodiment shown in FIG. 1.

FIG. 4 depicts a partial cross-sectional view of the distal end of the embodiment of FIG. 1.

FIG. 5 illustrates the embodiment of FIG. 1 in operative position relative to an eye.

FIG. 6 shows an enlarged cross-sectional view of the distal portion of the present invention in an operative position relative to an eye.

FIG. 7 depicts in an enlarged cross-sectional view an alternative embodiment of the present invention utilizing a micro-needle array in a retracted or non-operative position.

FIG. 8 shows the embodiment of FIG. 7 with the micro-needle array in an operative or extended position.

FIG. 9 illustrates the embodiment of FIG. 7 in an operative position relative to an eye.

FIG. 10 illustrates in an enlarged cross-sectional view an alternative embodiment of the present invention relative to an eye wherein a porous pad is utilized for the delivery of a therapeutic agent to the eye.

FIG. 11 shows in an enlarged cross-sectional view an alternative embodiment of the present invention relative to an eye wherein a porous pad is utilized for the delivery of a therapeutic agent to the eye, with the diffusion of the drug enhanced by iontophoresis.

### DETAILED DESCRIPTION OF THE INVENTION

The following discussion describes multiple embodiments of the present invention, each of which is provided for the delivery of a therapeutic agent for the treatment of macular degeneration.

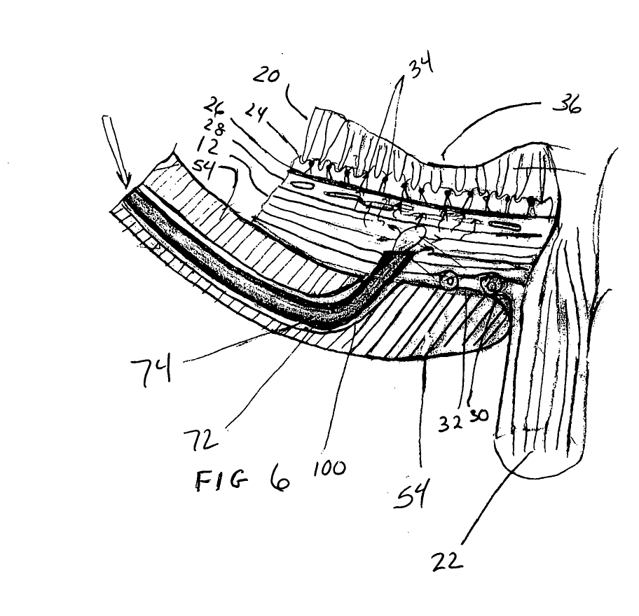

An embodiment 50 for providing macular degeneration therapy in accordance with the present invention is schematically shown in FIGS. 1-6 generally, with FIGS. 5-6 illustrating the invention relative to an eye. System 50 includes a handle 52 and a elongated hollow probe 54 with a proximal end 56 and a distal end 58. Probe 54 preferably is curved to negotiate the curved surface of the eyeball 60 and is sized to fit between a patient's sclera 12 and the eye lid 62.

More specifically, probe 54 includes a portion 64 and a curved probe portion 66. The curved probe portion is configured to conform to the curvature of an eye 10, thus reducing the likelihood of bruising or exertion of undue force on an eye during a procedure. The radius of curvature of the curved probe portion 66 may be in the range of about 12 mm to about 13 mm. Portion 66 may further include an eye-conforming and engaging surface 68 to further ease stresses on an eye during a procedure. It will be understood that the portion 64 may be configured as being substantially straight as illustrated, though it may take on other configurations if desired. It will also be understood that portion 64 may take on such lengths as appropriate.

Probe 54 has an outer wall 70 defining an elongated hollow interior space or passage 72 within which a drug delivery apparatus such as a needle 74 is disposed and travels between retracted and extended positions. Needle 74 includes proximal end 76 and distal end or needle tip 78.

Proximal end 76 of needle 74 is fluidly connected to a therapeutic agent reservoir 80. The reservoir is fluidly connected to the probe through the handle 52 as illustrated, though other known forms of fluid connections to the probe would also be appropriate. As schematically illustrated, the reservoir takes the form of a micro-injection system or syringe 82 with a plunger 84 movable within a chamber 86 containing the therapeutic agents. The microinjection system provides a predetermined therapeutic concentration of therapeutic agents discussed below at a predetermined rate and duration of infusion. Other systems known to the art capable of providing a controlled delivery of therapeutic agents may be used in accord with the present invention.

System 50 may also include a mechanism 90 appropriate for moving the needle 74 between its retracted and extended positions. The proximal needle end 76 is attached to a needle position adjustment mechanism 90. As illustrated in the Figures, mechanism 90 may take the form of a mechanical two-position mechanism known to the art. A two-position mechanism 90 such as that shown may be capable of moving the needle from a proximally reposed position wherein the needle end 78 is retracted within the probe 54 to a distally reposed position wherein the distal needle tip 78 penetrates the sclera 12. Stated otherwise, a two-position mechanism 90 has two positions along the hollow probe 54 as indicated by double-headed arrow 92. A first or proximal position is a passive position in that the needle 74 is in a retracted position within the probe 54. The second or distal position is an “active” position in that the needle penetrates sclera 12 by the tip 78. One advantage to using a two-position mechanism such as that shown is that it is a relatively simple mechanical mechanism. Another advantage is that the end 70 of the needle 68 may only be advanced a limited distance beyond the outer surface 68 of the probe interfacing with the sclera 12, thus substantially reducing the likelihood of damage to the eye by advancing the needle end 78 too great a distance into the eye. Other mechanisms capable of providing reciprocal motion of predetermined distances known to the art may be used in accord with the present invention.

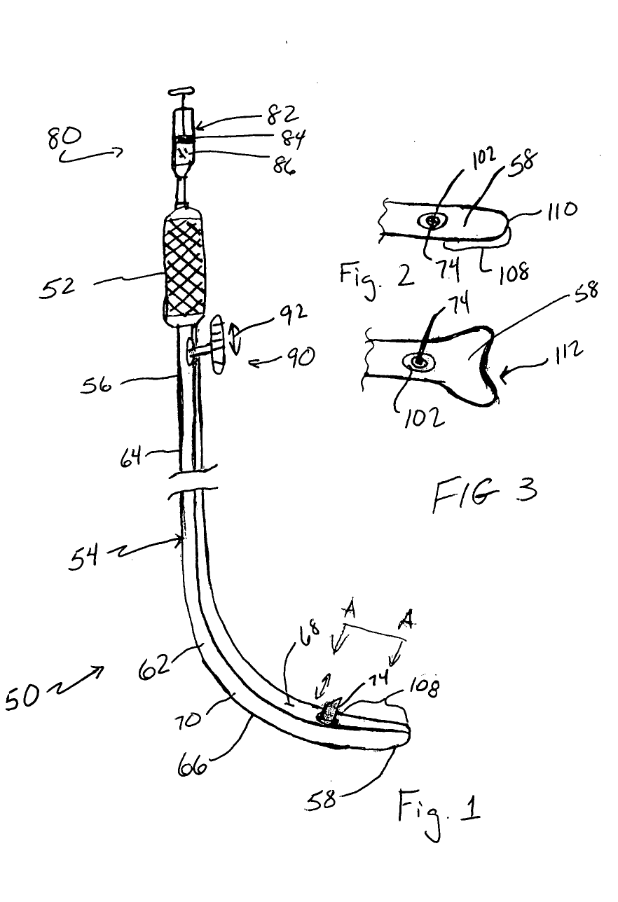

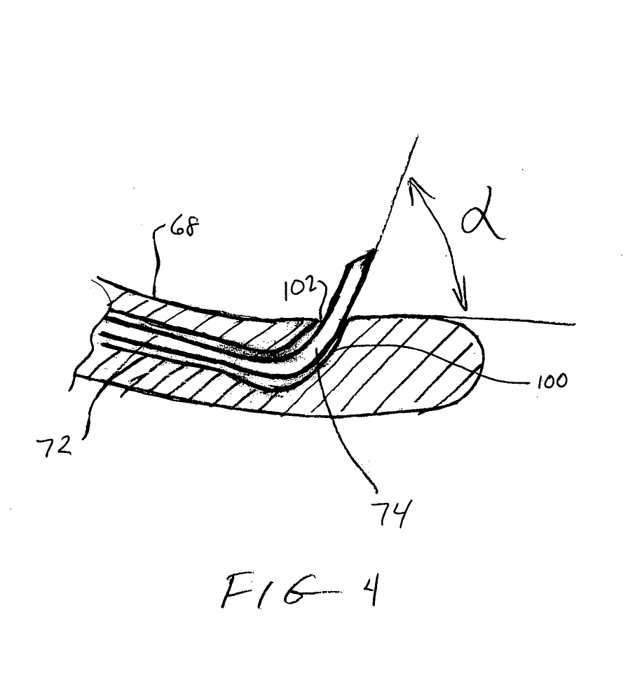

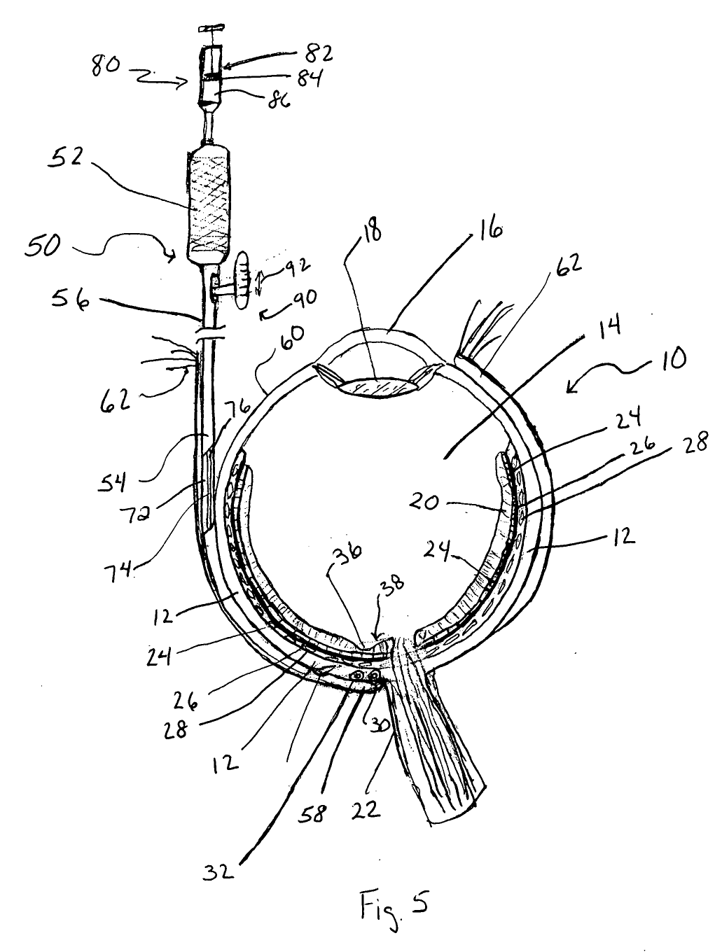

Referring to FIGS. 4 and 6, it will be observed that the passage 72 inside the probe 54 generally follows the longitudinal axis of the probe until near the distal end 58 thereof, where it may include a curved portion 100 that is adapted to bend the needle 74 in the direction of the sclera 12. That is, the curved portion 100 causes the needle 74, when advanced to turn in the direction of the sclera and exit the probe 54 through an opening 102 in the eye surface conforming surface 68. As the needle tip 78 exits the probe, it will form an angle α with surface 68. The angle α can be in the range of 0°<α90° and is preferably greater than about 60°. Stated otherwise, in the curved probe portion 66 of the probe 54 the passage 72 will follow a curve conforming to the tissue profile provided by the surface 60 of the eye and will then curve toward to eye so as to redirect the needle tip 78 thereto. Needles made of materials such as nitinol may be used in accord with the present invention.

Referring specifically to FIGS. 1-3 and 5-6, it will be observed that the probe 54 may include a positioning portion 108 extending from the needle opening 102 to the distal probe end 58 that is disposed between the sclera 12 and the eye socket. Positioning portion 108 may include a rounded or blunt end 110 as seen in greater in FIG. 2. Alternatively, probe distal end 58 may have a “C” or cupped configuration 112 as seen in FIG. 3. Positioning portion 108 should be appropriately sized so that during a procedure the distal end 58 of probe 54 may engage the optic nerve 22 and thus dispose the needle opening 102 relative to the eye such that the needle tip 78 will not be at risk of puncturing the artery 30 or vein 32 as best seen in FIG. 6. For adults, positioning portion 108 should have a length of about at least 5 mm.

The inside channel of the needle 74 will be in fluid communicating with the reservoir 80 and will deliver therapeutic agents or drugs via the sharp tip 78 of needle 74 directly into sclera 12. From the site of injection the injected (or infused) solution of lipase diffuses across sclera 12, choroid 28, through the Bruch's membrane 26 until it encounters the blood-ocular barrier created by tight junctions 34 in the apical domain of the RPE membrane. Injected lipase or any other drug will not diffuse beyond the tight junctions 34 comprising the blood-ocular barrier. Thus the retina 20 is protected from possible toxicity of the lipase solution and any concomitant substances that may be added to the solution.

According to a method of providing therapy for macular degeneration in accord with the present invention, the probe 54 is temporarily implanted between the eye socket and the sclera 12. The needle 74, fluidly connected to the reservoir 80, is forced by the needle advancement and retraction mechanism 90 into the advanced position, in which it penetrates up to 0.5 to 0.75 of the sclera thickness (about 0.9-1 mm). Subsequently, a therapeutic solution is injected into sclera. After a predetermined time of infusion the needle will be retracted back into the passage 72 of probe 54 by mechanism 90 and the probe 54 will be removed from the eye socket.

The number and duration of the treatments depends on the severity of the degeneration, the age of the patient and any other circumstance relevant to providing such treatment. As indicated, the procedure is a minimally invasive procedure that may be provided on the outpatient basis under local anesthesia.

The sclera 12 does not have a well-defined boundary with the choroid 28, so solutions of even very high molecular weight substances can relatively freely diffuse from the sclera, across the choroid and then to the RPE 24. Further diffusion will be halted, as noted earlier the junctions 34 forming the blood-ocular barrier formed by the RPE 24. Consequently, the therapeutic drug solution provided to the sclera by system 50 may freely diffuse from the end 78 of the needle 74 as far as RPE apical membrane, where it will be stopped by the tight junctions 34 of the blood-ocular barrier. Because Bruch's membrane 26 lies between the choroid 28 and the RPE 24, it is subject to delivery of therapeutic agents to purge it of neutral lipid deposits. In accordance with the teachings of the present invention, the delivery of lipase and/or other substances identified below, as a therapeutic agent to Bruch's membrane will dissolve neutral lipids into free fatty acids, which will then diffuse into capillaries in the choroid 28 and be carried away by the blood stream.

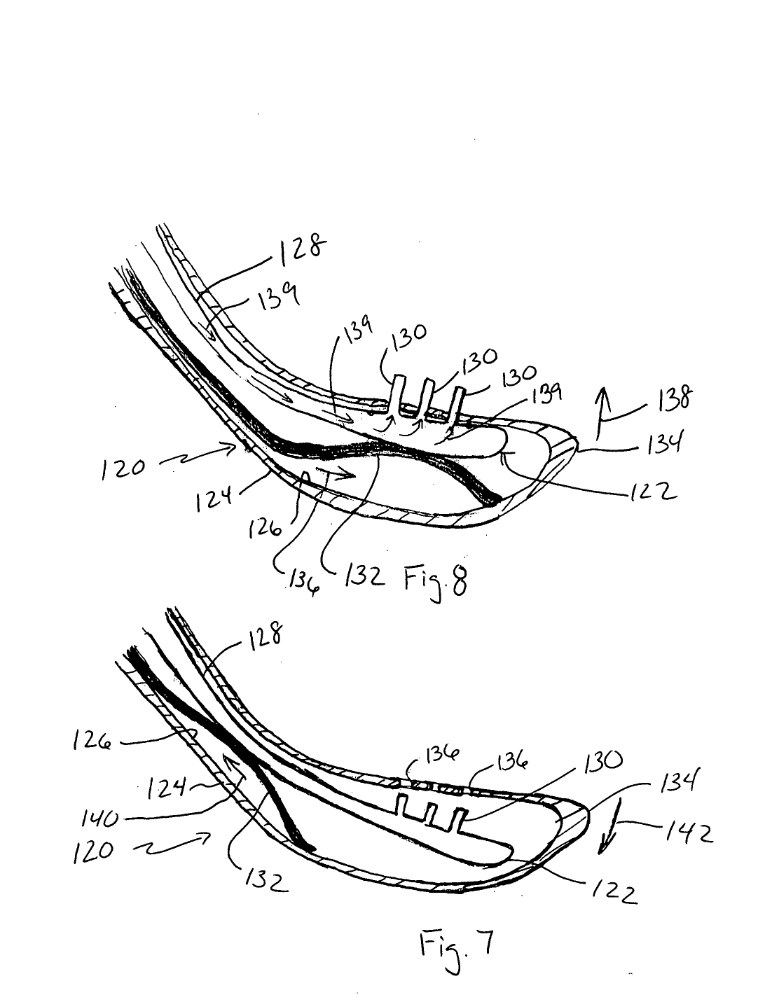

In FIGS. 7-9 another embodiment of an apparatus for drug delivery into sclera 12 is shown. In this embodiment 120 of the present invention the single needle 74 of apparatus 50 is replaced by a micro-needle array 122. Thus, referring to FIG. 7, embodiment 120 includes a probe 124 having a passage 126. Housed within the passage 126 is a tube 128 that is fluidly connected to the reservoir 80, which provides a solution of therapeutic agents such as lipase to the micro-needle array 122. Micro-needle array 122 comprises a plurality of needles 130 fluidly connected to tube 128.

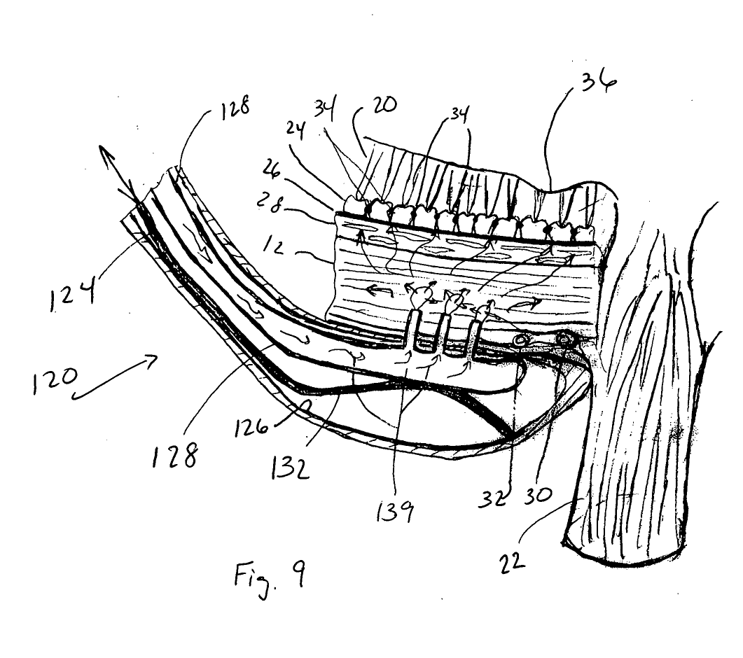

The micro-needle array 122 may assume one of two possible positions: a retracted, inactive position and an extended or advanced, active position in which the micro-needles 130 of the array 122 penetrate the sclera 12. To move the array 122 between the inactive and active positions, a spring 132 may be provided. Spring 132 is attached to an advancement mechanism such as mechanism 90. The array 122 may be moved from its inoperative position to its operative position by advancing the spring 132 towards the distal end 134 of the probe 124 as indicated by arrow 136. As seen in the Figures, the spring 132 engages the array 122 so as to move it towards the eye as indicated by arrow 138 until the needles 130 extend out through one or more openings 136 in the probe 124. Stated otherwise, in the inoperative position spring 132 is in compressed against the tube 128 as seen in FIG. 7. Advancing the spring 132 toward distal probe end 134 enables the spring to expand against the array 122 and push the array 122 toward the eye so that the needles 130 extend beyond the probe surface and enter the eye as seen in FIGS. 8-9. Infusion of the therapeutic agents from the reservoir 80 may then begin as indicated by arrows 139. Retraction of the spring as indicated by arrow 140 will allow the array to return to its inoperative position as indicated by arrow 142.

Needles 130 may have an outer diameter of 40-100 microns and a height about 120-200 microns, thus providing the appropriate extension beyond the surface of the probe and depth of penetration into the sclera 12. To provide the necessary restoring action of the array 122 such that it returns to its inoperative position, thus withdrawing the needles 130 from the sclera 12 and enabling the probe 120 to be removed from the eye socket, tube 128 may be made of steel or other known materials providing the appropriate restoring action. Thus, the probe 120 provides apparatus that enables one or more needles to be moved at substantially a 90° angle directly into the sclera 12.

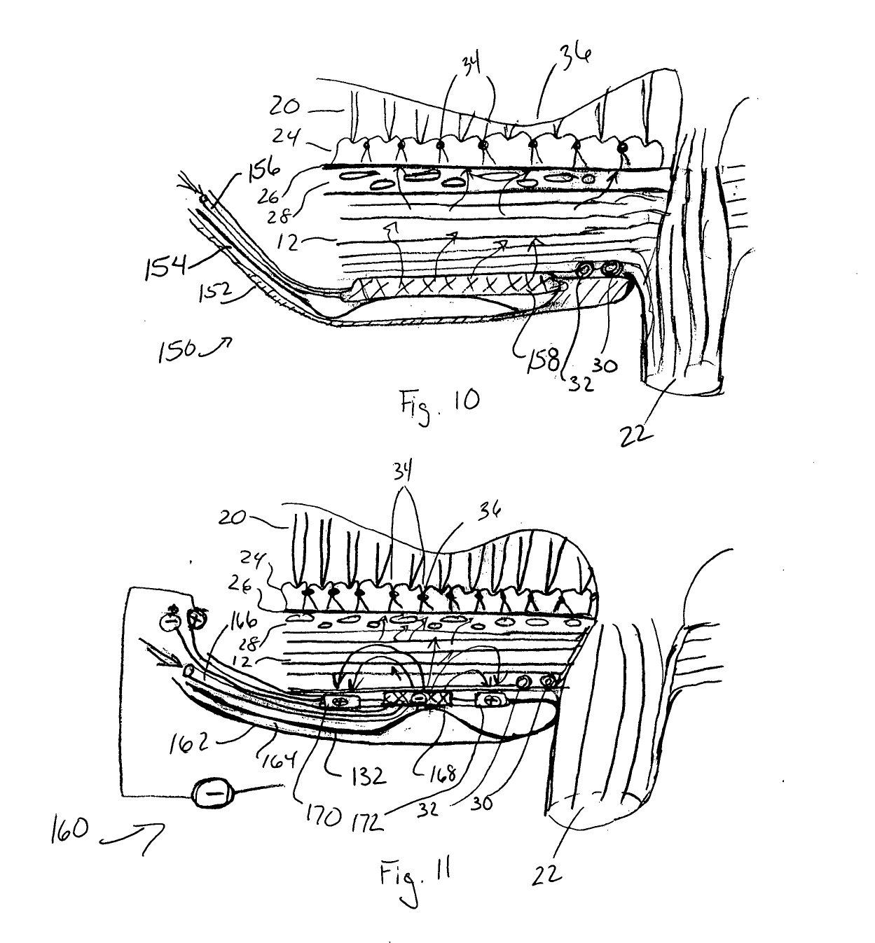

FIG. 10 illustrates another implementation 150 of the present invention. Apparatus 150 comprises a probe 152 having an interior passage 154 therein. Passage 154 houses a tube 156 in fluid communication with reservoir 80 and a porous pad 158. Pad 158 may also take inoperative and operative positions wherein it is housed within the passage 154 of the probe 152 and placed into contact with the sclera 12, respectively. A spring 132 operated as previously described may be used to move the pad 158 between the two positions.

In this embodiment the delivery of the therapeutic agents into the sclera is performed by diffusion. The tube 156 may be fluidly connected with a microinjection system or other known system of drug delivery by diffusion, for example, an osmotic pump.

In FIG. 11 another embodiment 160 of the present invention is shown. This embodiment is similar to apparatus 150 depicted in FIG. 10, but enhances delivery of the therapeutic agents into the sclera with iontophoresis. Thus, apparatus 160 includes a probe 162 having a passage 164 that houses a tube 166 connected to a reservoir 80 and one or more diffusion and conductive pads. As illustrated, each of the pads are both conductive and serve as diffusion pads, though separate pads could be provided for each purpose if desired. Thus, the apparatus 160 also includes one or more porous conductive pads 170 and 172 soaked with therapeutic agents and electrically connected to a negative electrode by wire 174 and one or more conductive pads 168 electrically connected to a positive electrode by wire 176. That is, the conductive pads are connected to positive and negative electrodes with wires 174 and 176 of a DC power source, not shown in the figure. Electric current passing between the pads enhances the drug delivery from the porous pads inside the sclera. If desired, a remote electrode outside the eye could also be used rather than providing both positive and negative electrodes adjacent to the sclera as shown.

Pads 168-172 may be brought into contact with sclera 12 by means of a spring 132 as previously described.

Generally speaking, the drugs may be delivered in bolus or by a prolonged infusion by apparatus and method in accord with the present invention. The drug delivery may be performed by an apparatus having just one needle or an array of micro-needles. To enhance the drug delivery an iontophoresis apparatus may be temporarily implanted on the posterior sclera.

The present invention, then, provides a method for treating macular degeneration including providing a reservoir of at least one therapeutic agent for dissolving lipids in Bruch's membrane; providing an elongate probe configured to engage the surface of the eye in the proximity of the optic nerve; providing a therapeutic agent delivery apparatus for delivering the therapeutic agent to the sclera; and delivering the therapeutic agent to the sclera. The elongate probe may include a therapeutic agent dispensation opening such as the opening for the needle, the micro-needle array, or the porous pads discussed previously for providing the therapeutic agent to the eye. A probe useful in the present invention may also comprise a probe positioning portion for positioning the therapeutic dispensing opening in close proximity to the fovea.

Regarding the therapeutic agents, the following discussion will focus on known substances, though it should be understood that any newly discovered substance that performs as described herein and that is safe for use may also be used with the present invention.

Lipase is active at the interfaces between the fat deposits and the aqueous phase. Delivered to the sclera, it will diffuse through the sclera's sponge-like tissue to Bruch's membrane, where it will interact with neutral fat deposits (triglycerides) to hydrolyze or transform them into free fatty acids and glycerol. The molecules of free fatty acids and glycerol are comparatively small and will diffuse around the space of Bruch's membrane and the choroid until they get into choroidal capillaries and be carried away by the blood stream. Freed from the neutral fat deposits, Bruch's membrane can sustain significantly more intensive metabolism between the RPE and choroidal capillaries providing nutrients and oxygen to retina and voiding wastes from the retina.

The whole lipase family: human, animal pancreatic lipases, co-lipases and bacterial or plant origin lipase may be used for the treatment. The lipase can be bought from multiple vendors, for example, at the time of the filing of this application, lipase is available from Calzyme Laboratories, Inc. located at 3443 Miguelito Court in San Luis Obispo, Calif. 93401.

Some additives may be used for enhancement of lipase activity or its stability in solution. As a provider of Ca²⁺ ions, calcium chloride (CaCl₂) is a known activator of lipase and may be used in combination with lipases for therapeutic injections into the sclera. Other salts that provide Ca²⁺ ions may also be used.

Bile salts are known for up to 5 fold increase of the lipase stability in solution. Using additives of bile salts is beneficial in another aspect: Bruch's membrane is clogged in part by deposits of cholesterol and the bile salts are capable of dissolving cholesterol and removing it from the membrane in blood stream.

Albumin, the most abundant protein in blood and a natural detergent, is known as a binder and carrier of free fatty acids in blood circulation. Added to the therapeutic solution of lipase and injected into sclera, it can bind the free fatty acids released by the lipase and carry them away into the choroid, thus avoiding any possible toxic action of the released free fatty acids on the RPE, Bruch's membrane and the choroid. A variety of natural and artificial detergents capable of binding fatty acids and carrying them in aquatic solution may be used instead of albumin. Long list of these detergents may be found in and selected from the product catalog of company EMD Biosciences, Inc, Calbiochem, located at 10394 Pacific Center Court San Diego, Calif. 92121, USA.

Thus, in accord with the present invention, the therapeutic agent may include lipase supplemented if desired with calcium chloride or other Ca²⁺ salts, bile salts, albumin, and/or other detergents capable of binding fatty acids and carrying them into aquatic solution.

The present invention has been described in language more or less specific as to the apparatus and method features. It is to be understood, however, that the present invention is not limited to the specific features described, since the apparatus and method herein disclosed comprise exemplary forms of putting the present invention into effect. The invention is, therefore, claimed in any of its forms or modifications within the proper scope of the appended claims appropriately interpreted in accordance with the doctrine of equivalency and other applicable judicial doctrines.

What is claimed is:

1. A minimally invasive therapeutic agent delivery system for treating macular degeneration, said system comprising:
a reservoir comprising a therapeutic agent for dissolving lipid waste deposits in at least Bruch's membrane;
an elongate probe, wherein said probe:
defines a passage therein;
is configured to conform at least in part to the curvature of the eye;
has a proximal probe end and a distal probe end including a distal probe opening;
a therapeutic agent delivery apparatus, said therapeutic agent delivery apparatus being:
fluidly connected to said reservoir;
configured to be disposed within said passage; and
movable between a retracted inoperative position within said probe and an extended operational position when said distal probe end is disposed adjacent the sclera of an eye suffering from macular degeneration
wherein movement of said delivery apparatus from the inactive to the operational position enables the therapeutic agents to be dispensed from said reservoir through said distal probe opening into the eye for the treatment of macular degeneration.

2. The system of claim 1 and further including a handle attached to said probe proximal end.

3. The system of claim 1 and further including a handle attached to said probe proximal end, wherein said reservoir is attached to said handle.

4. The system of claim 1 wherein said therapeutic agent delivery apparatus comprises an elongate needle.

5. The system of claim 4 wherein said probe distal end includes an eye-surface engaging surface configured to conform to the surface of the eye.

6. The system of claim 5 wherein said probe passage includes a portion conforming to the surface of the eye and a portion that angles toward the eye such that said distal probe opening is in said eye-surface engaging surface.

7. The system of claim 5 wherein said passage bends said needle when said needle is moved from its retracted to its extended position.

8. The system of claim 1 wherein said probe includes a probe positioning portion at said distal probe end for engaging the optic nerve and positioning said distal probe opening relative to the fovea of the eye.

9. The system of claim 1 wherein said therapeutic agent delivery apparatus comprises an array of micro-needles.

10. The system of claim 9 wherein said probe houses a spring within said passage, said spring being provided for moving said array from its inoperative position to its operative position.

11. The system of claim 9 wherein said array is movable between retracted and extended positions by a spring.

12. The system of claim 1 wherein said therapeutic agent delivery apparatus comprises a porous pad.

13. The system of claim 12 wherein said pad is movable between operative and inoperative positions by a spring.

14. The system of claim 1 wherein said therapeutic agent delivery apparatus comprises a plurality of porous pads and at least one conductive pad, said porous pads being electrically connected to the negative electrode of a power source and said conductive pad being electrically connected to the positive electrode of a power source, said electrical connections being provided to enhance diffusion of the therapeutic agent into the eye.

15. A method for treating macular degeneration comprising:
providing a reservoir of at least one therapeutic agent for dissolving lipids in Bruch's membrane;
providing an elongate probe configured to engage the surface of the eye in the proximity of the optic nerve;
providing a therapeutic agent delivery apparatus for delivering the therapeutic agent to the sclera;
placing the elongate probe in position to deliver the therapeutic agent to the eye; and
delivering the therapeutic agent to the sclera.

16. The method of claim 15 wherein the therapeutic agent is lipase.

17. The method of claim 15 wherein the therapeutic agent is a solution of lipase and at least one therapeutic agent selected from calcium chloride, bile salts, and albumin.

18. The method of claim 15 wherein the therapeutic agent is a solution of lipase and a detergent for binding free fatty acids released by the lipase.

19. The method of claim 15 wherein said therapeutic agent is injected into the sclera.

20. The method of claim 15 wherein said therapeutic agent is diffused into the sclera.

21. The method of claim 20 wherein the diffusion of the therapeutic agent into the sclera is enhanced by iontophoresis.

22. The method of claim 15 wherein the elongate probe includes a therapeutic agent dispensation opening for providing the therapeutic agent to the eye.

23. The method of claim 22 wherein the elongate probe includes a probe positioning portion for positioning the therapeutic dispensing opening in close proximity to the fovea.

24. The method of claim 15 wherein therapeutic agent delivery apparatus is movable between inoperative and operative positions for delivery of the therapeutic agent.

25. The method of claim 24 and further including maintaining the therapeutic delivery apparatus in the inoperative position until the probe is disposed relative to the eye for delivery of the therapeutic agent.

26. The method of claim 25 and including disposing the therapeutic delivery apparatus in an operative position for delivery of the therapeutic agent and further including disposing the therapeutic delivery apparatus in the inoperative position for withdrawal of the probe relative to the eye.
